(19) United States
(12) Patent Application Publication
(10) Pub. No.: US 2008/0181356 A1
(43) Pub. Date: Jul. 31, 2008

(54) CAMERA USED WITH CT SCANNER TO TAKE EXTERNAL IMAGES

(76) Inventors: Predrag Sukovic, Birmingham, MI (US); Neal Clinthorne, Ann Arbor, MI (US); Miodrag Rakic, Redondo Beach, CA (US); Joseph Webster Stayman, Ann Arbor, MI (US); David Phillipe Sarment, Ann Arbor, MI (US); Matt Jacobson, Ann Arbor, MI (US)

Correspondence Address: CARLSON, GASKEY & OLDS, P.C., 400 WEST MAPLE ROAD, SUITE 350, BIRMINGHAM, MI, 48009, US

(21) Appl. No.: 12/020,775

(22) Filed: Jan. 28, 2008

Related U.S. Application Data
(60) Provisional application No. 60/897,567, filed on Jan. 26, 2007.

(51) Int. Cl. A61B 6/00; H04N 7/15

(52) U.S. Cl. 378/4; 348/14.1; 348/E07.083

(57) ABSTRACT

A camera mounted to a CT scanner can be used to provide technical assistance relating to the CT scanner. The camera captures an image of an operator of the CT scanner, and the image is provided to a technician at an off-site technical support center. The camera can take external images of a patient while the CT scanner captures x-ray images of the patient, and a computer registers the images relative to each other. The camera can be used when the CT scanner is operating in a fluoroscopy mode, for motion tracking or motion correction of a three dimensional CT image, or for soft-tissue simulation of an area of the patient that is to undergo surgery.

### REFERENCE TO RELATED APPLICATIONS

This application claims priority to U.S. Provisional Patent Application No. 60/897,567 filed Jan. 26, 2007.

### BACKGROUND OF THE INVENTION

The present invention relates generally to a camera used with a CT scanner that assists in providing technical support for the CT scanner and that can take external images of a patient in a fluoroscopy mode, provide for motion correction, CT scanner calibration, or image simulation.

A CT scanner takes a plurality of x-ray images of a part of a patient to create a three dimensional CT image. A prior art CT scanner includes a plurality of cameras that take external images of the patient. The cameras are each at a fixed location and do not move during a CT scan. A computer generates a three dimensional external image from the external images taken by the cameras. The three dimensional external image can then be associated with the three dimensional CT image. The cameras are only used to take external images of the patient for association with the three dimensional CT image. It would be beneficial if cameras could be used for other purposes.

If an operator of a prior CT scanner system requires technical assistance, he telephones a technician at an off-site technical support center. A drawback to this type of communication is that the technician cannot see the operator.

### SUMMARY OF THE INVENTION

A CT scanner includes a gantry including an x-ray source and a detector. The x-ray source generates x-rays that are directed toward the detector that converts the x-rays to visible light and an array of photodetectors behind a converter to create an image. As the gantry rotates about a patient, the detector takes a plurality of x-ray images at a plurality of rotational positions, creating a three dimensional CT image.

In one example, a camera is located on a gantry of the CT scanner and is capable of taking images of an operator of the CT scanner. The images can be a photographic image or a video image. The images of the operator are sent by a computer to an off-site technical support center, where they are viewable by a technician to provide technical assistance. The off-site technical support center can also include a camera that is capable of taking images of the technician that can be viewed by the operator.

In another example, the camera takes external images of the patient when the CT scanner is operating in a fluoroscopy mode. The external images taken by the camera and the x-ray images taken by the CT scanner are provided to the computer and registered relative to each other.

In another example, the camera can take external images of the patient that can be used for motion tracking or motion correction of the three dimensional CT image. The computer evaluates both the external images and the x-rays images to determine if the patient has moved during the CT scan and uses this information to correct for the motion of the patient in the part of the three dimensional CT image that correlates to the time of movement.

In another example, the camera can be used with the CT scanner for soft-tissue simulation of an area of the patient to predict a post-operative appearance of the patient.

These and other features of the present invention will be best understood from the following specification and drawings.

### BRIEF DESCRIPTION OF THE DRAWINGS

FIG. 1 schematically illustrates a first embodiment CT scanner that is contained in a room with a camera mounted to the CT scanner;

FIG. 2 illustrates the CT scanner of FIG. 1 with a part of a person received in the CT scanner;

FIG. 3 illustrates a second embodiment of the CT scanner; and

FIG. 4 illustrates a computer employed with the CT scanner.

### DETAILED DESCRIPTION OF THE PREFERRED EMBODIMENT

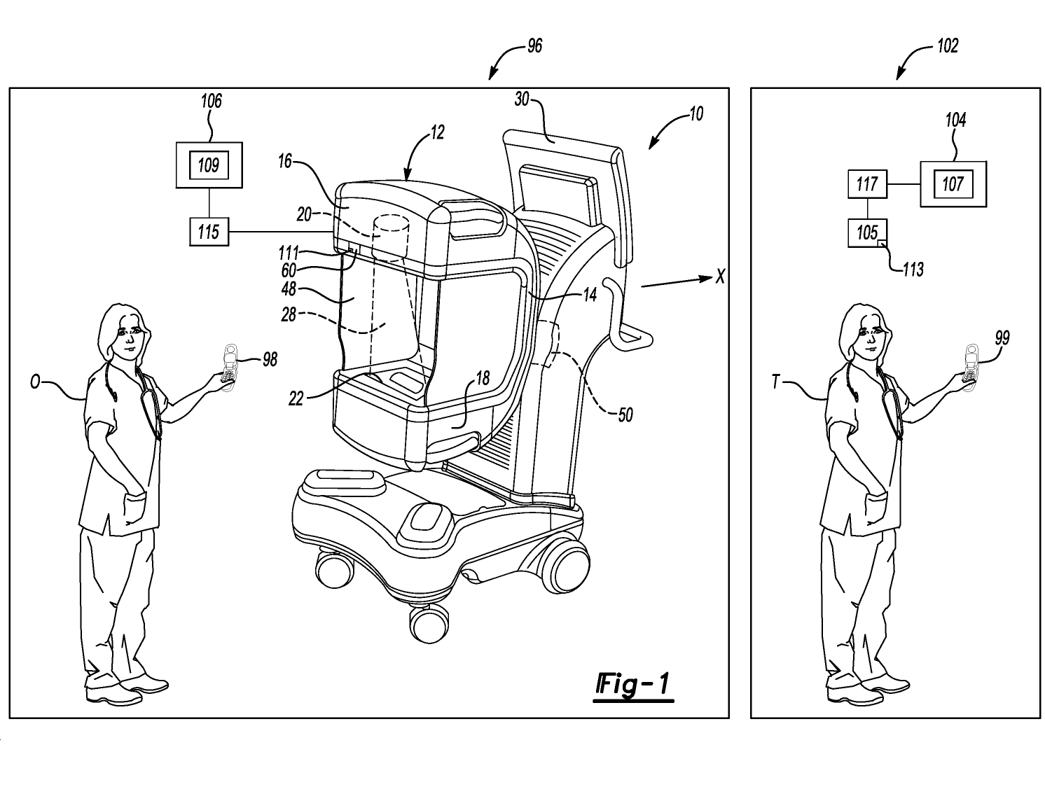

FIG. 1 illustrates a CT scanner 10 of the present invention contained in a room 96. The CT scanner 10 includes a gantry 12 that supports and houses components of the CT scanner 10. In one example, the gantry 12 includes a cross-bar section 14, and a first arm 16 and a second arm 18 each extend substantially perpendicularly from opposing ends of the cross-bar section 14 to form the c-shaped gantry 12. The first arm 16 houses an x-ray source 20 that generate x-rays 28. In one example, the x-ray source 20 is a cone-beam x-ray source. The second arm 18 houses a complementary flat-panel detector 22. The x-rays 28 are directed toward the detector 22 which includes a converter (not shown) that converts the x-rays 28 from the x-ray source 20 to visible light and an array of photodetectors behind the converter to create an image. As the gantry 12 rotates about the patient P, the detector 22 takes a plurality of x-ray images at a plurality of rotational positions. Various configurations and types of x-ray sources 20 and detectors 22 can be utilized, and the invention is largely independent of the specific technology used for the CT scanner 10.

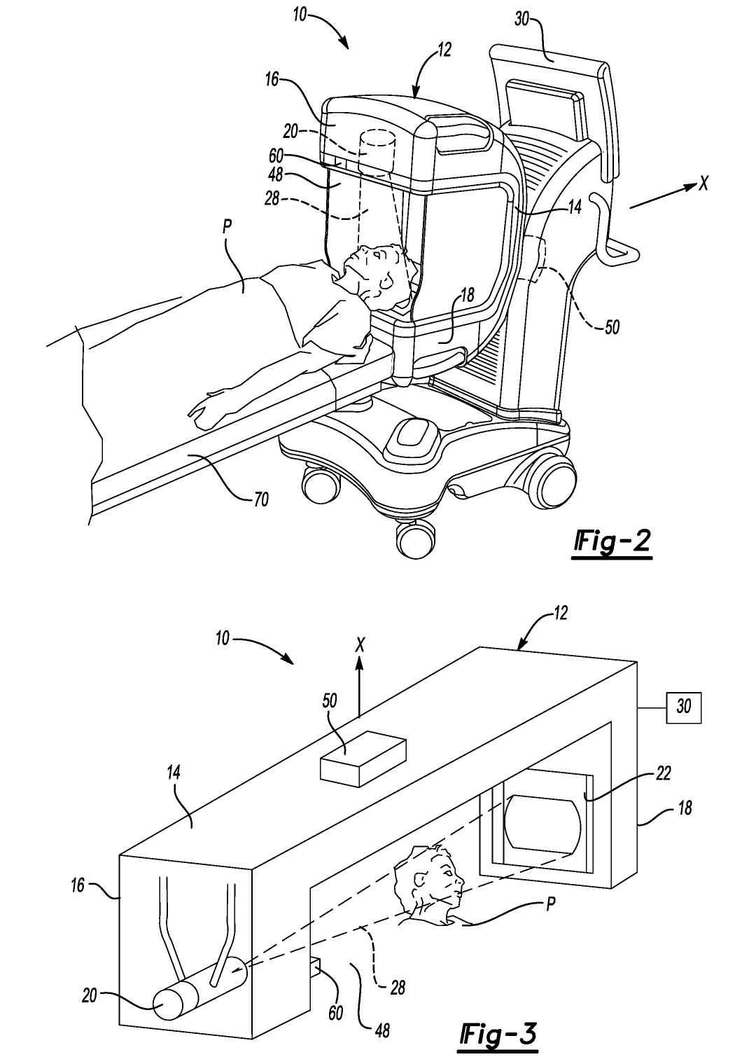

FIG. 2 illustrates the CT scanner 10 with a part of the patient P received in a space 48 between the first arm 16 and the second arm 18. A motor 50 rotates the gantry 12 about an axis of rotation X to obtain a plurality of x-ray images of the patient P at the plurality of rotational positions. The axis of rotation X is positioned between the x-ray source 20 and the detector 22. The gantry 12 can be rotated approximately slightly more than 360 degrees about the axis of rotation X. In one example, as shown in FIGS. 1 and 2, the axis of rotation X is substantially horizontal. In this example, the patient P is typically lying down on a table 70. Alternatively, as shown in FIG. 3, the axis of rotation X is substantially vertical. Typically, in this example, the patient P is sitting upright.

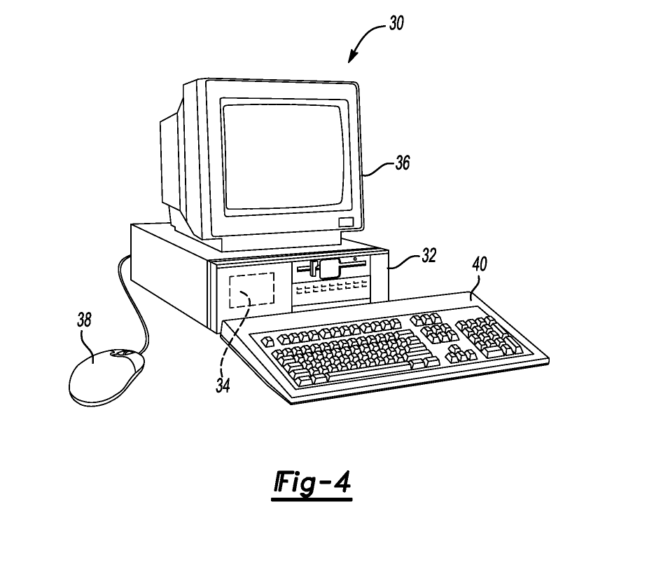

As shown schematically in FIG. 4, the CT scanner 10 further includes a computer 30 having a microprocessor or CPU 32, a storage 34 (memory, hard drive, optical, and/or magnetic, etc), a display 36, a mouse 38, a keyboard 40 and other hardware and software for performing the functions described herein. The computer 30 powers and controls the x-ray source 20 and the motor 50. The plurality of x-ray images taken by the detector 22 are sent to the computer 30. The computer 30 generates a three-dimensional CT image from the plurality of x-ray images utilizing any known techniques and algorithms. The three-dimensional CT image is stored on the storage 34 of the computer 30 and can be displayed on the display 36 for viewing.

Returning to FIG. 1, a camera 60 is located on the CT scanner 10. For example, the camera 60 can be attached to the gantry 12 of the CT scanner 10. In one example, the camera 60 is located on the first arm 16 near the x-ray source 20. However, the camera 60 can also be located on the second arm 18 near the detector 22. The camera 60 can be moved or pivoted relative to the CT scanner 10 such that the camera 60 is able to capture images of different objects in the room 96. In one example, the camera 60 is a digital camera. For example, the camera 60 is a web camera. However, any type of camera 60 can be employed. The camera 60 is capable of taking images of an operator O in the room 96. The images can be a photographic image (still image) or a video image (motion image).

In another example, the camera 60 can be located anywhere in the room 96, such as at a fixed location near the CT scanner 10. In this example, the camera 60 is capable of taking images of both the operator O and the CT scanner 10.

In one example, the camera 60 can be used to help provide technical support for the CT scanner 10. The camera 60 is associated with the computer 30 or an external computer 115. If the operator O of the CT scanner 10 needs technical assistance with the CT scanner 10, the camera 60 can provide images 107 of the operator O to a technician T at an off-site technical support center 102. In one example, the images 107 taken by the camera 60 are displayed on a display 104 at the off-site technical support center 102 for viewing by the technician T. The display 104 is associated with a computer 117. The images 107 can be provided to the technician T through the Internet. The technician T views the images 107 taken by the camera 60 to help diagnose and solve problems associated with the CT scanner 10 or to answer the questions of the operator O. In one example, the camera 60 includes a microphone 111 that allows the operator O to speak over the Internet. The off-site technical support center 102 also includes a microphone 113 associated with the computer 117 that also allows the technician T to speak over the Internet. In another example, the operator O and the technician T can speak on a telephone 98 and 99, respectively, while the technician T views the images 107.

The off-site technical support center 102 can also include a camera 105 associated with the computer 117 that is capable of taking images 109 of the technician T. The room 96 includes a display 106 associated with the computer 115 (or the display 36 of the computer 30) that allows the operator O to view the images 109 of the technician T captured by the camera 105. In one example, the images 109 are sent to the operator O through the Internet. The operator O can view the technician T, who can show the operator O how to service the CT scanner 10. In this example, the cameras 60 and 105 each include a microphone 111 and 113, respectively, and the operator O and the technician T can both speak over the Internet.

The camera 60 can also be used during a CT scan. The computer 30 can associate each of the external images taken by the camera 60 to one of the x-ray images taken by the CT scanner 10.

In one example, when the CT scanner 10 is operating in a fluoroscopy mode (the motor 50 is not rotating the gantry 12 about the axis of rotation X and the CT scanner 10 takes x-ray images), the camera 60 takes external images of the patient P when the CT scanner 10 takes internal x-ray images. For example, the external images and the x-ray images can be overlapped on the display 36

The operator O can manipulate the x-ray images stored on the computer 30. For example, the operator O can zoom in to view a specific area of the x-ray image on the display 36. If the operator O zooms out and away from an x-ray image, the x-ray image on the display 36 can change into the corresponding external image taken by the camera 60, providing the operator O with a reference image.

Alternately, both the x-ray images and the corresponding external images can be viewed side by side on the display 36. This allows the operator O to view actual external images of the external part of the patient P in addition to the x-ray images of the part of the patient P generated by the CT scanner 10. As the operator O manipulates the x-ray images on the display 36, the corresponding external images of the patient P taken by the camera 60 are also manipulated.

In another example, the camera 60 can take external images of the patient P during the CT scan that can be used for motion tracking or motion correction of the three dimensional CT image generated by the CT scanner 10. During the CT scan, the CT scanner 10 takes a plurality of x-ray images of the patient P. It is possible that the patient P may move during the CT scan. For example, the patient P might move between x-ray image number 45 and x-ray image number 46 taken by the CT scanner 10.

The computer 30 can use the external images to determine if the patient P has moved during the CT scan. The computer 30 evaluates both the external images and the x-rays images to determine if the patient P has moved during the CT scan. For example, the computer 30 evaluates both the external images and the x-ray images to determine that the patient P has moved between x-ray image number 45 and x-ray image number 46. The computer 30 uses this information to correct for the motion of the patient P in the part of the three dimensional CT image that correlates to x-ray image number 45 and x-ray image number 46.

In another example, the camera 60 can be used with the CT scanner 10 for soft-tissue simulation of an area of the patient P. For example, the area of the patient P can be a facial area having facial deformities that will undergo surgery. During a pre-operative CT scan, the CT scanner 10 takes a plurality of x-ray images of the area of the patient P to generate a pre-operative three dimensional CT image, and the camera 60 takes a plurality of external images of the area of the patient P to generate a pre-operative three dimensional external image.

Information about the type of surgery and the desired movement of bones in the facial area during surgery is programmed into the computer 30. Based on the pre-operative three dimensional CT image, the pre-operative three dimensional external image and the programming, the computer 30 predicts and simulates the appearance of the patient P after surgery. The predicted appearance of the patient P after the surgery can be simulated and displayed on the display 36 to give an indication of how the patient P will appear after surgery.

The foregoing description is only exemplary of the principles of the invention. Many modifications and variations are possible in light of the above teachings. It is, therefore, to be understood that within the scope of the appended claims, the invention may be practiced otherwise than using the example embodiments which have been specifically described. For that reason the following claims should be studied to determine the true scope and content of this invention.

What is claimed is:

1. A scanner comprising:
a gantry including a first arm section and a second arm section;
an x-ray source mounted to the first arm section;
an x-ray detector mounted to the second arm section, wherein the x-ray detector takes a plurality of x-ray images; and
a camera that is capable of capturing an image of an operator of the scanner.

2. The scanner as recited in claim 1 wherein the camera is one of a digital camera and a web camera.

3. The scanner as recited in claim 1 wherein the scanner is a CT scanner.

4. The scanner as recited in claim 1 further including a display at an off-site location that displays the image captured by the camera and a computer that transmits the image to the display.

5. The scanner as recited in claim 1 wherein the camera includes a microphone that allows the operator to communicate with a technician at the off-site location.

6. The scanner as recited in claim 1 further including another camera at the off-site location that is capable of taking another image of a technician and another display near the scanner, wherein a computer transmits the another image to the another display.

7. The scanner as recited in claim 6 wherein the another camera includes a microphone that allows the technician to communicate with the operator.

8. The scanner as recited in claim 1 further including a motor that rotates the gantry about an axis of rotation, wherein the x-ray detector takes the plurality of x-rays images as the gantry rotates.

9. The scanner as recited in claim 1 further including a computer that generates a three dimensional CT image from the plurality of x-ray images and stores the three dimensional CT image.

10. The scanner as recited in claim 1 wherein the camera captures an external image of a patient and a computer associates the external image with an associated x-ray image of the patient.

11. The scanner as recited in claim 10 wherein the camera captures the external image when the scanner is operating in a fluoroscopy mode.

12. The scanner as recited in claim 10 wherein the computer evaluates both the x-ray image and the external image to determine movement of the patient during a scan and corrects for movement of the patient.

13. The scanner as recited in claim 10 wherein the scanner captures a plurality of pre-operative x-ray images and the camera captures a plurality of pre-operative external images during a pre-operative scan, and the computer simulates a post-operative external image of the patient based on the plurality of pre-operative x-ray images, the plurality of pre-operative external images, and a type of procedure programmed into the computer.

14. A scanner comprising:
a gantry including a first arm section and a second arm section;
an x-ray source mounted to the first arm section;
an x-ray detector mounted to the second arm section, wherein the x-ray detector takes a plurality of x-ray images of a patient;
a camera that captures a plurality of external images of the patient; and
a computer that associates each of the plurality of x-ray images to one of the plurality of external images.

15. The scanner as recited in claim 14 wherein the scanner is a CT scanner.

16. The scanner as recited in claim 14 wherein the camera is mounted to the first arm section

17. The scanner as recited in claim 14 wherein the camera is one of a digital camera and a web camera.

18. The scanner as recited in claim 14 wherein the camera captures the at least one external image when the scanner is operating in a fluoroscopy mode.

19. The scanner as recited in claim 14 wherein the computer evaluates both the plurality of x-ray images and the plurality of external images to determine movement of the patient during a scan and corrects for movement of the patient.

20. The scanner as recited in claim 14 wherein the scanner takes a plurality of pre-operative x-ray images and the camera takes a plurality of pre-operative external images, the computer uses the plurality of pre-operative x-ray images to form a pre-operative three dimensional x-ray image and the plurality of pre-operative external images to form a pre-operative three dimensional external image, and the computer simulates a post-operative external image of the patient based on the pre-operative three dimensional x-ray image, the pre-operative three dimensional external image, and a type of procedure programmed into the computer.

21. A method of capturing an image, the method comprising the steps of:
capturing an image of an operator of a scanner with a camera that is mounted to the scanner, wherein the operator is in a room; and
transmitting the image of the operator to an off-site location.

22. The method as recited in claim 21 wherein the scanner is a CT scanner.

23. The method as recited in claim 21 further including the step of mounting the camera to a gantry of the scanner.

24. The method as recited in claim 21 further including the step of transmitting sound generated in the room to the off-site location.

25. The method as recited in claim 21 further including the step of transmitting an image of a technician at the off-site location to the room.

26. The method as recited in claim 21 further including the step of displaying the image at the off-site location to provide technical support.

26. A method of capturing an image of a patient, the method comprising:
obtaining a plurality of x-ray images of a patient;
obtaining a plurality of external images of the patient; and
associating each of the plurality of x-ray images to one of the plurality of external images.

27. The method as recited in claim 26 further including the step of mounting the camera to a gantry of a scanner.

28. The method as recited in claim 26 further including the step of operating a scanner in a fluoroscopy mode.

29. The method as recited in claim 26 further including the step of evaluating the plurality of x-ray images and the plurality of external images to determine movement of the patient during a scan and correcting for movement of the patient in a three dimensional image generated from the plurality of x-ray images.

30. The method as recited in claim 26 further including the steps of performing a pre-operative scan to generate a pre-operative three dimensional x-ray image and a pre-operative three dimensional external image, programming a type of procedure into a computer and simulating a post-operative external image of the patient based on the pre-operative three dimensional x-ray image, the pre-operative three dimensional external image and the type of procedure.
